(19) United States
(12) Patent Application Publication
(10) Pub. No.: US 2026/0344708 A1
(43) Pub. Date: Nov. 5, 2026

(54) COMPOSITIONS AND METHODS FOR THE MANAGEMENT OF NON-COPEPOD PARASITIC DISEASE IN AQUACULTURE SYSTEMS

(71) Applicant: CAN TECHNOLOGIES, INC., Wayzata, MN (US)

(72) Inventors: David A. COOK, Coon Rapids, MN (US); Alex Kalathil EAPEN, Medina, MN (US); Padmakumar B. PILLAI, Germantown, MD (US); Jorge Eduardo PINO MARAMBIO, Utrecht (NL); Stanko SKUGOR, Oslo (NO)

(73) Assignee: CAN TECHNOLOGIES, INC., Wayzata, MN (US)

(21) Appl. No.: 19/124,453

(22) PCT Filed: Oct. 26, 2023
(86) PCT No.: PCT/US2023/077914

§ 371 Date: Apr. 25, 2025

Related U.S. Application Data
(60) Provisional application No. 63/381,369, filed on Oct. 28, 2022.

(51) Int. Cl. A23K 50/80; A23K 10/30; A23K 20/121; A61K 31/34; A61K 36/58

(52) U.S. Cl. CPC A23K 50/80; A23K 10/30; A23K 20/121; A61K 31/34; A61K 36/58

(57) ABSTRACT

The present disclosure provides a method for reducing, preventing, or controlling a non-copepod parasitic infection or infestation in a fish population. The method includes providing a fish feed including a neem extract rich in azadirachtin A, the neem extract including from 15 wt. % to 33 wt. % of azadirachtin A and administering to one or more fish the fish feed including the neem extract rich in azadirachtin A, where the fish feed provides a concentration from 0.01 mg to 5.0 mg azadirachtin A per kg body weight per day to the one or more fish. Other aspects are also provided herein.

### CROSS-REFERENCE TO RELATED APPLICATIONS

This application claims the benefit of U.S. Provisional Application No. 63/381,369, filed Oct. 28, 2022, and entitled “COMPOSITIONS AND METHODS FOR THE MANAGEMENT OF PARASITIC DISEASE IN AQUACULTURE SYSTEMS,” which is incorporated by reference herein in its entirety.

### FIELD OF THE INVENTION

The present disclosure is directed to compositions and methods for the management of parasitic disease in fish. In particular, the present disclosure is directed to compositions and methods for administering a pest control agent through a fish feed or pest control agent composition to manage non-copepod parasitic infections or infestations in fish populations.

### BACKGROUND

There are multiple harmful parasites that can target fish in aquaculture and can cause dysregulated immune reactions in fish during an infection or infestation. The dysregulation of the immune system can include the suppression of protective, beneficial immune and physiological responses in parallel with the exaggeration of non-protective or harmful immune responses. Such dysregulation can cause a negative immune response whereby the immune system can be stopped, prevented, or decreased in effectiveness when faced with an immune challenge. In addition, overactivation of non-protective immune responses can be directly damaging for a host's tissues, which can lead to immunopathology in the host. By way of example, the salmon louse, Lepeophtheirus salmonis, is an ectoparasite that has adapted evolutionarily to attach to salmon and remain unharmed by the fish's immune system. Without wishing to be bound by any particular theory, it is believed that L. salmonis can modulate and evade the host immunity through the secretion of one or more molecules, including proteins, lipids, and the like to induce a suppressive effect on the protective, beneficial immune responses of fish. Such a down regulation of the protective host response can lead to opportunistic infection by other parasites, bacteria, viruses, protozoa, amoeba, and the like, which can in turn lead to an upregulation of non-protective or harmful immune responses. Many viral parasites modulate host immune responses to create an environment that favors progression of infection. In some cases, the down regulation of the protective host response can itself lead to an upregulation of the non-protective or harmful immune responses. Furthermore, overstimulation of non-protective or harmful immune responses can have numerous adverse effects in fish at the physiological, histological, biochemical, and enzymatic levels. By way of example, the energetic cost of a non-protective immune or physiological response may interfere with the fish's ability to clear toxic compounds, deal with oxidative stress, heat stress or other environmental assaults.

Various viruses and bacteria known to infect fish are thought to cause an upregulation of non-protective or harmful immune responses. An upregulation can lead to and activation or overactivation of the host immune response in fish and can lead to undesirable inflammatory side effect that can cause tissue damage (i.e., immunopathology), decreased animal welfare, a loss in farm productivity, and even death in the fish. By way of example, the Ichthyophthirius multifiliis can infect salmon and lead to significant economic losses for fish farmers.

There are various mechanisms available to manage parasitic diseases, including various synthetic medicinal agents, acid wash techniques, and vaccines. Such agents can be difficult to implement and be expensive in the long term. Additionally, resistance to various medicinal agents continues to be a problem in the field. Thus, a need exists to provide easy-to-use management methods that can be administered to fish to manage non-copepod parasitic disease by using simple and cost-effective means.

### SUMMARY

The present disclosure provides a method for reducing, preventing, or controlling a non-copepod parasitic infection or infestation in a fish population including providing a fish feed including a neem extract rich in azadirachtin A, the neem extract including from 15 wt. % to 33 wt. % of azadirachtin A and administering to one or more fish the fish feed including the neem extract rich in azadirachtin A, where the fish feed provides a concentration from 0.01 mg to 5.0 mg azadirachtin A per kg body weight per day to the one or more fish.

In an aspect, the method further includes where the non-copepod parasitic infection or infestation is caused by one or more parasitic organisms including Amyloodinium ocellatum, Cardicola forsteri, Cryptocaryon irritans, Diplectanum spp., Gyrodactylus salaris, Henneguya salminicola, Ichthyophthirius multifiliis, Kudoa thyrsites, Myxobolus cerebralis, Neoparamoeba perurans, Saprolegnia parasitica, Sparicotyle chrysophrii, Trichodina spp., or Zeylanicobdella arugamensis.

In an aspect, the method further includes where the fish feed is administered to the farmed fish for at least 11 days.

In an aspect, the method further includes where the fish feed is administered to the farmed fish for at least 14 days.

In an aspect, the method further includes where the concentration of azadirachtin A includes a concentration in an amount effective to increase efficacy of azadirachtin A against a non-copepod parasitic infection or infestation as compared to fish fed a diet lacking the neem extract rich in azadirachtin A.

In an aspect, the method further includes where the neem extract rich in azadirachtin A is administered to the fish at a concentration from 1.5 mg to 2.5 mg azadirachtin A per kg body weight per day.

In an aspect, the method further includes where the neem extract rich in azadirachtin A is administered to the fish at a concentration from 2.6 mg to 5.0 mg azadirachtin A per kg body weight per day.

In an aspect, the method further includes where the fish feed further includes one or more components including antibacterial agents, antifungal agents, antiviral agents, antiparasitic agents, or antiprotozoal agents.

In an aspect, the method further includes where the fish feed is administered to species of fish belonging to one or more families including Cyprinidae, Cichlidae, Pangasiidae, Sciaenidae, Serranidae, Carangidae, Sparidae, Lateolabracidae, Moronidae, Mugilidae, Cypriniformes, Latidae, Eleotridae, Tilapiini, and Salmonidae.

In an aspect, the method further includes where the pest control agent is configured to produce an inhibitory effect including one or more of an antiparasitic effect, an antibacterial effect, an antiviral effect, an antifungal effect, or an antiprotozoal effect.

In an aspect, the method further includes where the neem extract rich in azadirachtin A does not comprise neem oil.

In an aspect, the method further includes where the pest control agent is provided in the fish feed to the pests in an amount sufficient to modulate the behavior of the pests.

In an aspect, the method further includes where modulating the behavior of the pests includes one or more of a change in feeding habits, a change in feeding patterns, a change in appetite, a change in mobility patterns, or a change in mating patterns as compared to pests found on control animals not fed a pest control agent.

In an aspect, the method further includes where the change in feeding patterns can include a decrease in feeding patterns.

In an aspect, the method further includes where the change in appetite can include a decrease in appetite.

In an aspect, the method further includes where the change in mobility can include a decrease in mobility.

In an aspect, the method further includes where the change in mating patterns can include a decrease in mating patterns.

In an aspect, the method further includes where the change in mating patterns can include a decrease in the number of viable pests present on the fish or in the fish habitat as compared to pests found on control fish not fed a pest control agent.

In an aspect, the method further includes where the neem extract rich in azadirachtin A is obtained by a method including the steps of providing neem seeds; crushing the neem seeds;
extracting azadirachtin from the crushed seeds with water; adding a second extraction solution that includes a non-aqueous solvent which is not miscible with water and has a higher solubility of azadirachtin than water or a surfactant having a turbidity temperature between 20 °C and 80 °C; and recovering the concentrated azadirachtin from the second extraction solution.

The present disclosure provides a method for reducing, preventing, or controlling a non-copepod parasitic infection or infestation in fish including providing a pest control agent composition including a pest control agent including neem extract rich in azadirachtin A and administering the pest control agent composition to one or more fish for from 1 to 20 days during an infection or infestation, where the concentration of azadirachtin A administered to the fish through the pest control agent composition is from 0.01 mg to 5 mg azadirachtin A per kg body weight per day.

### BRIEF DESCRIPTION OF THE FIGURES

Not applicable.

### DETAILED DESCRIPTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

The present disclosure provides fish feeds and pest control agent compositions, containing one or more pest control agents for reducing, preventing, or controlling a parasitic infection or infestation in fish or a fish population. Various pests can cause dysregulation of a protective immune response or a protective physiological response within a host fish, and they can modulate the immune responses and physiological responses to their benefit. Some parasitic species can benefit by exposing a host to anti-inflammatory mediators to dampen the host's protective immune responses or protective physiological responses. Some parasitic species benefit by stimulating non-protective immune and non-protective physiological responses, which in turn suppresses activation of protective immune and protective physiological responses. Some parasitic species can exert their modulatory pressures long enough on a host fish such that the host fish's immune response results in harmful immunomodulatory or inflammatory effects for the fish long term.

Fish modulated to the advantage of parasites are less likely to be able to activate protective immune responses or protective physiological responses, such as Type 1 immune pathways. By way of example, the presence of some parasites, such as sea lice, can induce the production of antibodies by fish. However, the antibodies produced do not confer immune protection to the host and actually can drain the host of valuable resources in favor of providing a hospitable environment for the sea lice. Similarly, some parasites can upregulate the production of protective mucus found on the surface of the skin of fish. Salmon produce mucus as a physical and chemical protective barrier against invading pathogens. Certain parasites can take advantage of this protective physiological response by upregulating the volume of mucus produced by the fish, which in turn can provide an abundant food source for the parasites.

The pest control agents herein can be administered to fish and can be effective at modulating the host fish's immune response or physiological response in either the presence or absence of a parasite. The modulation of an immune response or physiological response can be exerted by the pest control agents prior to, after, or during an infection or infestation. The modulation of an immune response or physiological response can occur by decreasing the dysregulation of the immune response or the physiological response as caused by various parasites during an infection or infestation. In some aspects, the pest control agents can stimulate protective immune responses or suppress harmful immune responses in the fish. By stimulating protective immune responses and suppressing harmful immune responses, the pest control agents herein can contribute directly or indirectly in reducing, preventing, or controlling an infection or infestation caused by the pests described herein. In some aspects, the pest control agents herein can have a direct antiparasitic effect on parasites feeding on their hosts that have ingested the pest control agents be reducing the ability of the parasites to immunomodulate their hosts. The pest control agents can induce an immunomodulatory effect and provide anti-inflammatory effects by lessening the parasitic load in the fish or population of fish such that the host's immune responses and physiological responses can return to a pre-dysregulated state. In addition, the pest control agents can exert effects on the host immune and physiological pathways that can contribute to a positive outcome of a pest infection or infestation. For example, the pest control agents may promote effective antimicrobial immune responses, suppress non-protective and harmful immune responses, stimulate antioxidant defenses, detoxification pathways, repair of tissue damages mediated by own immune response or that caused by the pathogen and promote wound repair.

Modulation of an immune response or physiological response by pest control agents in host fish can include various immunomodulatory effects in the fish. The immunomodulatory effects can include a reduction or prevention of various immunopathology caused by a dysregulated immune response of physiological response. The immunopathology can include tissue damage caused by an exaggerated immune response, such as decrease in heart muscle damage, skeletal muscle damage, and the like.

The present disclosure provides fish feeds, pest control agent compositions, and pest control agents for reducing, preventing, or controlling one or more non-copepod parasites in fish and a fish population. As used herein, the term “copepod” refers to a group of crustaceans found in fresh water and in seawater, and which have one or more parasitic phases of their life cycle including various species of sea lice. As used herein, the term “non-copepod” refers to a group of parasites that are not copepods and are known to infect or infest fish species not copepods.

As used herein, the term “infection” can refer to a condition where a pestilent organism, including the various pests defined elsewhere herein, can invade any internal or external portion of a host organism's body such that the host organism experiences harm, and where the pestilent organism uses components of the host organism to sustain itself, reproduce, or colonize the host organism.

As used herein, the term “infestation” can refer to the presence of an abnormally large number of pests as defined herein, where the pests are concentrated in a region in numbers that can cause damage or disease through infection of a host organism.

As used herein, the term “pest” can refer to any organism that is detrimental to the health, value, or appearance of another organism. The term pest can include, but is not to be limited to, one or more of various parasites including worms, helminths, and flukes; one or more species of bacteria; one or more viruses; one or more type of fungi; and various protozoa (e.g., amoeba).

As used herein, the term “parasite” can refer to one or more species of ectoparasite or endoparasite. As used herein, the term “endoparasite” can refer to organisms that inhabit one or more internal niches of another organism. For example, an endoparasite can inhabit one or more of the tissues, organs, or systems of a host organism. For example an endoparasite can inhabit the gut, blood, or both, of a host organism. As used herein, the term “ectoparasite” can refer to organisms that inhabit or occupy an external niche of another species. For example, an ectoparasite can inhabit or occupy the surface of a host species. In the case of fish, ectoparasites can inhabit the skin of the fish where they sometimes lodge between scales, and they further can feed off of the mucus, blood, skin, gills, muscle, or any combination thereof.

As used herein, the term “pest control agent” can refer to an agent for reducing, preventing, or controlling an infection or infestation caused or contributed to by one or more pests. In various aspects, the pest control agents described herein can refer to an agent for reducing, preventing, or controlling an infection or infestation caused by one or more endoparasites or ectoparasites.

### Target Fish

The fish feeds provided herein can be fed as a fish feed diet or used to feed any fish that is susceptible to infection or infestation by one or more pest. For example, the fish feed can be used in aquaculture as a component of a diet fed to any farmed fish including, for example, commercially relevant fish species. For example, the fish feeds provided herein can form part of diet fed to any of freshwater fish, brackish fish, or saltwater fish. The fish feeds can be used as a component of a diet fed to any species belonging to the families Cyprinidae, Cichlidae, Pangasiidae, Sciaenidae, Serranidae, Carangidae, Sparidae, Lateolabracidae, Moronidae, Mugilidae, Cypriniformes, Latidae, Eleotridae, Tilapiini and Salmonidae. As such, the fish feeds herein can be used to feed species belonging to any of the genera within these families and in particular, those species that are farmed for human or animal consumption. For example, and without limitation, the fish feeds described herein can be used to feed species belonging to the genera Salmo and/or Oncorhynchus. In particular, the fish feeds herein can be used to control pests in populations of wild or farmed salmon or trout species, including, for example, any of Atlantic salmon (Salmo salar), Pacific salmon, Char, or Rainbow trout. Moreover, the fish feed can be used as a pest control agent for other fish species within the aquaculture industry such as sea bass, bream, grouper, pompano, and tuna, as well as in the pet and decorative fish industries, for example for pest control in koi (Cyprinus rubrofuscus) and goldfish (Carassius auratus).

### Target Parasites

Pests of the target fish herein can include non-copepod parasites that can cause a number of diseases in fish. The non-copepod parasites that are suitable for managing with the pest control agents described herein can include one or more of Amyloodinium ocellatum, Cardicola forsteri, Cryptocaryon irritans, Diplectanum spp., Gyrodactylus salaris, Henneguya salminicola, Ichthyophthirius multifiliis, Kudoa thyrsites, Myxobolus cerebralis, Neoparamoeba perurans, Saprolegnia spp. including Saprolegnia parasitica, Sparicotyle chrysophrii, Trichodina spp., or Zeylanicobdella arugamensis. These organisms can all cause one or more clinical manifestations of disease that can decrease the wellbeing of the fish, affect the edible muscle quality of the fish, can lower farm productivity by decreasing the fish available for market, increase mortality in the fish populations, and can lead to significant economic losses for farmers.

The dinoflagellate ectoparasite Amyloodinium ocellatum is known to cause amyloodiniosis in farmed and aquarium fish alike. Amyloodiniosis is characterized primarily as an infestation of the gills of fish, but can also infest the skin, fins, and eyes. Clinical symptoms of amyloodiniosis can include anorexia, decreased activity, labored breathing, and poor overall wellbeing in the fish.

The parasitic ciliate Ichthyophthirius multifiliis can cause the disease in fish often called “Ich.” I. multifiliis shows low host specificity and infests the interior of the organism on the epithelial layers of the gills, skin, and fins. I. multifiliis can cause significant damage to the skin, gills, and fins of its host, and thus the fish experience distress and lead to anorexia, respiratory distress, discoloration and color spotting, inactivity, scratching against objects, and upside-down swimming. If left uncontrolled, I. multifiliis can lead to 100% mortality rates.

The ciliate Cryptocaryon irritans is known to cause marine white spot disease in wild and farm-raised fish. C. irritans organisms feed on the skin, eyes, and gills of fish and can deteriorate the function of these tissues. The marine white spot disease caused by C. irritans can lead to whiteish nodules on the fish and to an overproduction of mucus, discoloration of the skin, anorexia, hyperactivity, poor overall wellbeing, and respiratory distress in the fish.

The organisms of the genus Diplectanum are parasites that infect the gill of fish species. Those Diplectanum suitable as targets for the pest control agents herein can include but are not to be limited to, Diplectanum aculeatum, Diplectanum aequans, Diplectanum banyulense, Diplectanum belengeri, Diplectanum bocqueti, Diplectanum chabaudi, Diplectanum copiosum, Diplectanum dollfusi, Diplectanum femineum, or Diplectanum flagritubus. The Diplectanum spp. can infest fish by feeding on the gills and causing diplectanosis in the fish. Fish with diplectanosis can present with damaged gill tissue, respiratory distress, poor overall wellbeing, and secondary infection due to viruses and bacteria.

The salmon fluke, Gyrodactylus salaris, is a parasite species that lives on the surface of the body of many freshwater fish. G. salaris feeds on the body of many freshwater fish and can cause skin wounds and open the fish to co-infection with one or more additional pests.

Cardicola forsteri is a blood fluke that can infect bluefin tunas. C. forsteri can cause severe branchitis, or swelling of the gills, and myocarditis in fish and can lead to high mortality rates in fish populations.

Kudoa thyrsites is a myxozoan parasite that infects the muscle of a broad range of marine fishes and has a global distribution. K. thyrsites can cause nodules or cysts within the muscle tissue of the fish. The infection can lead to an exaggerated immune response that can lead to tissue degeneration whereby the tissue becomes soft due to enzymatic digestion of the muscle fibers. Fish infected with K. thyrsites can become damaged to the point that they are no longer of sufficient quality for marketing to consumers, thus causing significant losses to farmers.

The myxosporean Henneguya salminicola (also known as Henneguya zschokkei) can infect or infest fish and cause a disease called milky flesh or tapioca disease. Milky flesh or tapioca disease as caused by H. salminicola, can be characterized in that the muscle of the fish is intermittently spotted with small, white cysts filled with the spores of the organism. The milky flesh disease can disrupt farm productivity, because while generally the disorder is harmless, it causes a loss in economic profit due to unsightly flesh caused by the disorder.

The myxosporean Myxobolus cerebralis causes whirling disease in various fish species. Whirling disease can be characterized as a chronic disease that infects salmon, and particularly juvenile salmon, and causes severe deformities in the cartilage, bone, and muscle of the fish. When the parasite infects the cartilage of the fish its presence can create pressure against the spinal cord and cause a characteristic swimming pattern in the form of a whirl. The disease can be particularly uncomfortable for the fish and can lead to severe economic loss for farmers.

The protozoan Neoparamoeba perurans (synonym, Paramoeba perurans) causes amoebic gill disease in various fish species. The protozoan attaches to the gills of fish and causes tissue damage through increased mucus production and multifocal patches of swollen tissue. Fish experience respiratory distress due to the gill damage. The disease causes extreme distress in fish and can lead to significant economic losses for fish farms.

The organism Sparicotyle chrysophrii is a parasite that infects the gills of various fish species. Once the organisms attaches to the gills it causes tissue damage that can lead to respiratory distress, anemia, and death. The disease causes extreme distress in fish and can lead to significant economic losses for fish farms.

The single-celled flagellates of the genus Trichodina can infect or infest various fish, including those common to ornamental fishponds such as koi and goldfish. Trichodina attaches to the skin and gills of fish and causes damage to these tissues as well as respiratory distress, anorexia, and loss of scales.

The fungus of the genus Saprolegnia spp., including Saprolegnia parasitica, can infect or infest various fish types can cause saprolegniasis. Saprolegniasis can be characterized in fish by the presence of greyish-white patches having filamentous mycelium on the body or fins of fish. The disorder can cause tissue damage and in extreme cases, death.

Zeylanicobdella arugamensis is a species of marine leeches that can parasitize a number of fish species reared in aquaculture. Z. arugamensis attaches to the skin, gills, fins, and mouth surfaces of various fish species and causes wounds in areas of attachment. Wounds caused by attachment and feeding of Z. arugamensis can predispose the fish to secondary infection with other pests.

### Fish Feeds

The present disclosure provides fish feeds to be used as fish diets in aquaculture applications. It will be appreciated that the fish feeds herein can include suitable types of fish feed specific for a given fish species. The fish feeds can be used as a component of a diet fed to any species belonging to the families Cyprinidae, Cichlidae, Pangasiidae, Sciaenidae, Serranidae, Carangidae, Sparidae, Lateolabracidae, Moronidae, Mugilidae, Cypriniformes, Latidae, Eleotridae, Tilapiini and Salmonidae. In various aspects, this disclosure provides a fish feed or fish feed diet for species within the family Salmonidae. The fish feeds provided herein can be used to feed wild fish or farmed fish. In various aspects, both wild fish and farmed fish can be fed simultaneously. Further, the fish feed can be used to feed freshwater fish or salt water (e.g., marine) fish, or both.

The fish feeds of the present disclosure can be produced using a base feed formulation that is a solid feed or a liquid feed using raw materials that can be chosen based on the application in which it is to be used and on the fish species. In various aspects, the fish feed is a solid fish feed. In other aspects, the fish feed is a liquid fish feed. In other aspects, the fish feed can include both a solid fish feed component and a liquid fish feed component. Fish feeds in solid form can include pellets, extruded nuggets, steam pellets, flakes, tablets, powders, and the like. In various aspects, the base feed can include a base feed pellet. In some aspects, the base feed pellet can include a porous matrix distributed throughout. Fish feeds in liquid form can include aqueous solutions, oils, oil and water emulsions, slurries, suspensions, and the like. In various aspects, a solid fish feed can further include one or more oils disposed on the surface or distributed throughout the fish feed.

The fish feeds herein can include a number of different ingredients or raw materials that can sustain life, growth, and reproduction of the fish. The fish feeds can include any substrate that is edible to fish. For example, an edible substrate can provide a source of nutrition to the fish or can be an inert substrate with no nutritive value to the fish. In various aspects, the fish feeds herein can include feeds that are either nutritional fish feeds or non-nutritional feeds. Nutritional fish feeds can include a nutritional food stuff formulated for fish as part of its diet as the main source of nutrition, growth, and reproduction. Suitable nutritional fish feeds can include one or more of proteinaceous material as a source of proteins, peptides, and amino acids; carbohydrates; and fats, as described below. Non-nutritional fish feeds can include any substrate that is edible to fish but does not provide nutrition to sustain life, growth, or reproduction. In various aspects, the nutritional or non-nutritional fish feeds herein can include one or more compounds designed to alter the quality, quantity, or appearance of a fish and fish tissue. For example, a nutritional or non-nutritional fish feed can include a carotenoid compound to improve the appearance (e.g., color) of the muscle tissue. By way of example, the carotenoid compound can include compounds such as astaxanthin.

The fish feeds herein can include a complete fish feed. A complete fish feed can include a feed for fish that is compounded to be fed as the sole ration and that can maintain life, promote growth, and sustain reproduction without any additional substances being consumed except water. Complete fish feeds can include compounded mixtures containing various energy sources such as carbohydrates, proteins, and fats. In various aspects, the fish feeds herein can include at least a protein and a starch. Additional ingredients can be included, such as vitamins and minerals as necessary to support the life, growth, and reproduction of fish. A complete fish feed can include ingredients such as, but not limited to, fish meal, poultry meal, plant meal, vegetable meal, corn meal, corn gluten meal, soy meal, soy protein concentrate, single cell protein, insect meal, algae meal, algae oil, krill meal, krill oil, meat meal, blood meal, feather meal, starches, tapioca starch, wheat, wheat gluten, guar meal, guar protein concentrate, peas, pea protein concentrate, pea starch, beans, faba beans, sunflower meal, vegetable oil, canola oil, poultry oil, rapeseed oil, fish oil, soy oil, linseed oil, camelina oil, lecithin, macro-minerals, minerals, vitamins, amino acids, pigments, and any combinations thereof. It will be appreciated that the fish feeds herein can include fish meal that can include plant or animal derived matter. Any animal derived matter present in the fish meal can be derived from the same species of fish or a different species of fish (e.g., heterologous species or non-fish species).

The total protein content in the fish feed can be from 10 wt. % to 70 wt. %, from 15 wt. % to 65 wt. %, from 20 wt. % to 60 wt. %, or from 25 wt. % to about 55 wt. %. The total protein in the fish feed can be at least 10%, 15%, 20%, 25%, 30%, 35%, 38%, 40%, 42%, 44%, 46%, 48%, 50%, 52%, 55%, 60%, 65%, or at least 70% by weight, or any amount within a range of any of the forgoing. The total protein in the fish feed can be variable depending on the formulation, species, age, and intended use of the feed. It will be appreciated that the various protein requirements of the species of fish receiving the fish feed can be adjusted to meet the protein requirements of that species.

The protein in the fish feeds herein can be from any suitable source including, but not limited to, one or more of fish meal, land-animal protein (e.g., poultry meal), plant-based protein (e.g., vegetable meal), or any combinations thereof. The fish feed can include from 0% to 80%, from 10% to 80%, from 20% to 75%, from 30% to 70%, from 60% to 80%, or from 10% to 30%, or at least 10%, 20%, 30%, 40%, 50%, 60%, 70%, or 75% fish meal by weight, or any amount within a range of any of the forgoing. The fish feed can include from 0% to 80%, from 10% to 80%, from 20% to 75%, from 30% to 70%, from 60% to 80%, or from 10% to 30%, or at least 10%, 20%, 30%, 40%, 50%, 60%, 70%, or 75% land-animal protein by weight, or any amount within a range of any of the forgoing. The fish feed can include between 0% to 80%, from 10% to 80%, from 20% to 75%, from 30% to 70%, from 60% to 80%, or from 10% to 30%, or at least 10%, 20%, 30%, 40%, 50%, 60%, 70%, or 75% plant-based protein by weight, or any amount within a range of any of the forgoing.

Total fat (e.g., oil, fat, and/or lipids) in the fish feed can be from 5% to 50%, from 10% to 45%, from 15% to 40%, or from 20% to 35%. The total fat in the fish feed can be at least 5%, 10%, 15%, 20%, 25%, 30%, 35%, 40%, 45%, or at least 50% by weight, or any amount within a range of any of the forgoing. The total fat in the fish feed can be variable depending on the formulation, target fish species, and intended use of the fish feed. It will be appreciated that the various fat requirements of the species of fish receiving the fish feed and can be adjusted to meet the fat requirements of that species. Suitable fats for use herein can include, but are not to be limited to, those provided by canola oil, poultry oil, rapeseed oil, fish oil, soy oil, linseed oil, camelina oil, palm oil, lecithin, or any combinations or fractions thereof.

The moisture content of the fish feeds herein can vary depending on the contents and preparation method of the feed. In various aspects, the moisture content can be from 1% to 20%, from 2% to 18%, from 5% to 15%, or from 6% to 12% by weight.

The fish feeds herein can include one or more pest control agents. In various aspects, the one or more pest control agents can be present in the fish feed in an amount effective to produce an inhibitory effect on one or more pests, as will be described elsewhere herein. As such, the fish feeds herein can include pest control agents for controlling infections or infestations caused by one or more pests. In various aspects, the fish feeds herein can include pest control agents for controlling endoparasitic or ectoparasitic infections or infestations. Various pests suitable as targets for the pest control agents herein are described elsewhere.

Each pest control agent included within the fish feeds herein can be individually capable of controlling one or more of a parasitic, bacterial, viral, fungal, or protozoal infections or infestations. Therefore, it should be understood that any given pest control agent for use in the many aspects described herein can be referred to as exhibiting one or more inhibitory effects, including antiparasitic effects (e.g., anti-ectoparasitic, anti-endoparasitic), antibacterial effects, antiviral effects, antifungal effects, or antiprotozoal effects. In various aspects, the inhibitory effect can include an antiparasitic effect, where the antiparasitic effect can further include an anti-ectoparasitic effect, an anti-endoparasitic effect, or both. It will be appreciated that the inhibitory effects can result in reducing, preventing, or controlling the concentration and spread of the various parasitic, bacterial, viral, fungal, or protozoal organisms described herein. In various aspects, a pest control agent of the present disclosure can produce inhibitory effects against one or more pests including one or more effects for reducing, preventing, or controlling the concentration and spread of various endoparasites or ectoparasites. Reducing, preventing, or controlling the parasites can include complete prevention of infection or infestation in the fish population or on each fish, a reduction in the total number of parasites present in the fish population or on each fish, or controlling how many parasites are present in the fish population or on each fish according to local regulatory requirements. It will be appreciated that the inhibitory effects described herein can be measured against a population fish infected or infested with one or more pests that are fed a diet lacking the neem extract rich in azadirachtin A.

In other aspects, the inhibitory effect against the pests can include one or more of an anti-feedancy effect, an anti-molting effect, an antifertility and anti-fecundity effect, or an antiparasitic effect. As used herein, “anti-feedancy effect” can refer to an effect exerted by one or more pest control agents that stops or inhibits feeding by the pests resulting in their malnourishment, delayed development, prevention or delay of molting, and death. Malnourished parasites are also less likely to efficiently immunomodulate their hosts, and thus they are less capable evading host immunity. As used herein, the term “anti-molting” can refer to an effect exerted by one or more pest control agents that prevents or delays the process of molting in the pests. The process of molting occurs as pests grow and shed their exoskeletons from one life stage to the next and is controlled hormonally and neuronally, and the pest control agents herein can exert one or more anti-molting effects against the pests. As used herein, the terms “antifertility effect” and “anti-fecundity effect” are referred to collectively as an “antifertility and anti-fecundity effect” and can include one or more effects on male or female reproduction. It will be appreciated that the term “fertility” can refer to the actual number of offspring born to or eggs released from a female, and the term “fecundity” can refer to the biological potential for reproduction, and due to their close relationship the two terms as used herein can be used interchangeably unless otherwise noted. An antifertility and anti-fecundity effect can include a reduction in total gamete production in males and females, a complete or partial inhibition of viable egg production, a change in the anatomy and morphology of the gametes of males or females, a change in the potential for egg fertilization, and a reduction in the total number of gravid female pests.

In an administered form, the fish feeds herein can include an amount of pest control agent at from about 0.01-100 grams of pest control agent per kilogram fish feed (g/kg), about 90 g/kg fish feed, about 80 g/kg fish feed, about 70 g/kg fish feed, about 60 g/kg fish feed, about 50 g/kg fish feed, about 40 g/kg fish feed, about 30 g/kg fish feed, about 20 g/kg fish feed, about 1-10 g/kg fish feed, about 2-9 g/kg fish feed, about 3-7 g/kg fish feed, about 4-6 g/kg fish feed, or about 5 g/kg fish feed.

In various aspects, the fish feeds herein can include an amount of pest control agent and/or active ingredient in an amount effective to produce an inhibitory effect against one or more pests, including a concentration from about 0.01 g/kg fish feed, 0.05 g/kg fish feed, 0.1 g/kg fish feed, 0.2 g/kg fish feed, 0.3 g/kg fish feed, 0.4 g/kg fish feed, 0.5 g/kg fish feed, 0.6 g/kg fish feed, 0.7 g/kg fish feed, 0.8 g/kg fish feed, 0.9 g/kg fish feed, 1.0 g/kg fish feed. 1.25 g/kg fish feed, 1.5 g/kg fish feed, 1.75 g/kg fish feed, 2.0 g/kg fish feed, 2.25 g/kg fish feed, 2.5 g/kg fish feed, 2.75, g/kg fish feed, 3.0 g/kg fish feed, 5.0 g/kg fish feed. 5.25 g/kg fish feed, 5.5 g/kg fish feed, 5.75 g/kg fish feed, 6.0 g/kg fish feed, 6.25 g/kg fish feed, 6.5 g/kg fish feed, 6.75, g/kg fish feed, 7.0 g/kg fish feed, 7.0 g/kg fish feed. 7.25 g/kg fish feed, 7.5 g/kg fish feed, 7.75 g/kg fish feed, 8.0 g/kg fish feed, 8.25 g/kg fish feed, 8.5 g/kg fish feed, 8.75, g/kg fish feed, 9.0 g/kg fish feed, 9.25 g/kg fish feed, 9.5 g/kg fish feed, 9.75, g/kg fish feed, 10.0 g/kg fish feed, 15 g/kg fish feed, 20 g/kg fish feed, 25 g/kg fish feed, 30 g/kg fish feed, 35 g/kg fish feed, 40 g/kg fish feed, 45 g/kg fish feed, 50 g/kg fish feed, 55 g/kg fish feed, 60 g/kg fish feed, 65 g/kg fish feed, 70 g/kg fish feed, 75 g/kg fish feed, 80 g/kg fish feed, 85 g/kg fish feed, 90 g/kg fish feed, 95 g/kg fish feed, or 100 g/kg fish feed, or any amount within a range of any of the forgoing concentrations.

It will be appreciated that the aforementioned concentrations equate to an amount from about 0.001-10 weight percent (% w/w) total pest control agent to fish feed. In various aspects, the fish feeds herein can include an amount of pest control agent effective to produce an inhibitory effect against one or more pests including from 0.001% w/w, 0.0020% w/w, 0.003% w/w, 0.004% w/w, 0.005% w/w, 0.006% w/w, 0.007% w/w, 0.008% w/w, 0.009% w/w, 0.010% w/w, 0.020% w/w, 0.030% w/w, 0.040% w/w, 0.050% w/w, 0.060% w/w, 0.070% w/w, 0.080% w/w, 0.090% w/w, 0.10% w/w, 0.11% w/w, 0.12% w/w, 0.13% w/w, 0.14% w/w, 0.15% w/w, 0.16% w/w, 0.17% w/w, 0.18% w/w, 0.19% w/w, 0.20% w/w, 0.21% w/w, 0.22% w/w, 0.23% w/w, 0.24% w/w, 0.25% w/w, 0.26% w/w, 0.27% w/w, 0.28% w/w, 0.29% w/w, 0.30% w/w, 0.31% w/w, 0.32% w/w, 0.33% w/w, 0.34% w/w, 0.35% w/w, 0.36% w/w, 0.37% w/w, 0.38% w/w, 0.39% w/w, 0.40% w/w, 0.41% w/w, 0.42% w/w, 0.43% w/w, 0.44% w/w, 0.45% w/w, 0.46% w/w, 0.47% w/w, 0.48% w/w, 0.49% w/w, 0.50% w/w, 0.51% w/w, 0.52% w/w, 0.53% w/w, 0.54% w/w, 0.55% w/w, 0.56% w/w, 0.57% w/w, 0.58% w/w, 0.59% w/w, 0.60% w/w, 0.61% w/w, 0.62% w/w, 0.63% w/w, 0.64% w/w, 0.65% w/w, 0.66% w/w, 0.67% w/w, 0.68% w/w, 0.69% w/w, 0.70% w/w, 0.71% w/w, 0.72% w/w, 0.73% w/w, 0.74% w/w, 0.75% w/w, 0.76% w/w, 0.77% w/w, 0.78% w/w, 0.79% w/w, 0.80% w/w, 0.81% w/w, 0.82% w/w, 0.83% w/w, 0.84% w/w, 0.85% w/w, 0.86% w/w, 0.87% w/w, 0.88% w/w, 0.89% w/w, 0.90% w/w, 0.91% w/w, 0.92% w/w, 0.93% w/w, 0.94% w/w, 0.95% w/w, 0.96% w/w, 0.97% w/w, 0.98% w/w, 0.99% w/w, 1.0% w/w, 2.0% w/w, 3.0% w/w, 4.0% w/w, 5.0% w/w, 6.0% w/w, 7.0% w/w, 8.0% w/w, 9.0% w/w, or 10.0% w/w, or any amount within a range of any of the forgoing values.

In various aspects herein, the fish feeds can be administered to the fish having a concentration of pest control agent selected from the group including 0.05% w/w, 0.06% w/w, 0.07% w/w, 0.08% w/w, 0.09% w/w, 0.10% w/w, 0.11% w/w, 0.12% w/w, 0.13% w/w, 0.14% w/w, 0.15% w/w, 0.16% w/w, 0.17% w/w, 0.18% w/w, 0.19% w/w, 0.20% w/w, 0.30% w/w, 0.40% w/w, 0.50% w/w, 0.60% w/w, 0.70% w/w, 0.80% w/w, 0.90% w/w, or 1.0% w/w, pest control agent to fish feed or a range within any of the forgoing concentrations.

### Pest Control Agents

The pest control agents suitable for use in the fish feeds and pest control agent compositions herein can adversely affect pests that feed off their hosts. It will be appreciated that when a host fish has consumed the pest control agents as a component of their daily diet for a given duration, it can be transferred to the body of the pest when that pest takes a meal from the host. The pest control agents can include any functional agent or active agent that affects, facilitates, or contributes to the eradication or reduction of a pest infection or infestation of a fish or population of fish. Additionally, suitable pest control agents can alleviate or improve one or more of the symptoms associated with a pest infection or infestation, as a result of reducing, preventing, or controlling an infection or infestation. Pest control agents for use herein can be biologically active to one or more fish pests and for one or more fish species.

It will be appreciated that when the fish consume the pest control agents described herein, the pest control agent is systemically distributed throughout the tissues and fluids of the fish. Pests can be exposed to the pest control agents upon ingestion of the pest control agent through the skin, flesh, blood, mucus, mucous membranes, or other tissues of the host organism. Modulation of the pests, such as modulation of the pest behavior and life cycle occur to reduce, prevent, or control the pest infection or infestation in the fish. In various aspects, the pests can be repelled or killed by the pest control agents herein. Thus, the pest control agents herein can be provided to the pests in a fish feed or a pest control agent composition in an amount sufficient to modulate the behavior of the pests.

Modulation of the pests can have many effects on the pest population, including an ultimate reduction in the number of viable pests available to infect or infest the host fish. Modulation of the pests can include a modulation of the mortality of the pests. It will be appreciated that modulation of the mortality in the pests can include a decrease in the number of viable pests present on the fish or in the fish habitat. Modulation of the pests can further include modulation of pest behavior, including a change in feeding habits, a change in feeding patterns, a change in appetite, a change in mobility patterns, a change in swimming and migration patterns, a change in mating patterns, a change in development, a change in fertility, or any combination thereof, as compared to pests found on control fish not fed a pest control agent. The change in feeding patterns can include a decrease in feeding patterns. The change in appetite can include a decrease in appetite. The change in mobility can include a decrease in mobility. The change in swimming and migration patterns can include a decrease in swimming and migration due to lethargy and lack of energy. The change in mating patterns can include a decrease in mating patterns, which in turn can lead to a decrease in development or production of offspring. The change in development can include an inhibition of development due to an inhibition of the molting process leading to a decrease in development in the pests or a delay in development in the pests. The change in fertility can include an inhibition of or delay in egg production, an inability to produce viable eggs, or a reduction in the total number of gravid female pests.

In various aspects, modulation of the pests can include a change in development of the pests through their life cycle, including modulation of growth or progression through a particular life stage, modulation of growth or progression from one life stage to the next life stage (e.g., modulating molting), modulation of egg production, modulation of fertility, or any combination thereof. Modulation of growth or progression through a particular life stage can include halting the growth of the organism and preventing further physical development including a decrease in size or sexual development. In some aspects, modulation of growth or progression from one life stage to the next life stage can include preventing the pests from transitioning from one life stage to the next by inhibiting the molting process. Modulation of egg production can include decreasing the production of eggs by females, which can further result in a decrease in fertility of the adult females. Modulation of fertility can include decreasing the fertility of both female and male pests.

In various aspects, the administration of pest control agents to fish as described herein further can have a beneficial effect on the fish. The administration of the pest control agents can impart a beneficial effect by improving fish welfare by reducing the parasitic load, or total number of parasites, in a given environment around the fish. The administration of the pest control agents can impart a beneficial effect by a reduction in the overall mortality within a fish population by lessening or reducing the impact of a parasitic infection or infestation on the fish population. The administration of the pest control agents can impart a beneficial effect by minimizing or altogether eliminating the impact on the quality and quantity of fish flesh within the fish population.

The fish feeds described herein can include, or be supplemented with, one or more pest control agents. Where a fish feed includes at least two or more different pest control agents, each pest control agent can be individually active (or biologically active) and capable of modulating one or more of the behavior, development, or fertility of a pest. Alternatively, the pest control agents can be a component of a pest control agent composition that can be fed separately to fish. Each pest control agent can be individually effective against one or more different pests as described herein.

Pest control agents suitable for use in the fish feeds and pest control agent compositions herein can include one or more active agents, including synthetic or natural agents. The one or more synthetic or natural agents can include agents classified as an active pharmaceutical ingredient, a veterinary medicinal product, and the like. In some aspects, the active agent for the pest control agents herein can be obtained from a plant belonging to the genus Azadirachta. The pest control agent can be obtained or extracted from Azadirachta indica—a tree commonly known as the “Neem” tree. Extracts prepared from plants belonging to the genus Azadirachta (e.g., Azadirachta indica) can include potent terpenoid compounds, including one or more azadirachtinoids. The azadirachtinoids include azadirachtin compounds such as azadirachtin A, azadirachtin B, azadirachtin D, azadirachtin E, azadirachtin F, azadirachtin G, azadirachtin H, azadirachtin I, azadirachtin K, and/or other azadirachtin variants. The extracts from plants belonging to the genus Azadirachta can also include many other components in various quantities. In some aspects, the extracts can include additional compounds such as the limonoids salannin, nimbin, deacetylsalinin, and 6-desacetylnimbin. In various aspects, the extracts can further include one or more azadirachtinins.

As used herein, the term “azadirachtin” can refer to the collective term applied to a large group of active compounds and is intended to encompass not only all naturally occurring variants or derivatives of azadirachtin, including but not limited to azadirachtins A, B, D, E, F, G, H, I, K, but also all synthetic variants, fragments, analogues, and derivatives thereof. In this regard, it will be appreciated that any azadirachtin variants, fragments, derivatives, or analogues for use herein should be functional, in that they exhibit at least one inhibitory effect as described.

Azadirachtin can be obtained or extracted from any part of the Azadirachta indica plant including, for example, the leaves, stems, bark, fruit, seeds, or any combinations thereof by one or more extraction processes. Suitable methods of extraction can include techniques that exploit mechanical pressing of neem seeds (i.e., kernels) and the use of non-polar solvents. Various solvent extraction techniques exploiting alcohol or an aqueous extraction process, mechanical pressing, and non-polar extraction methods can be used to produce azadirachtin A-rich pest control agents for use herein and are described in U.S. Pat. Nos. 4,556,562; 5,695,763; and 11,096,404; the contents of which are incorporated herein by reference in their entirety.

For example, azadirachtin can be effectively recovered from the seeds of the Neem tree. An exemplary method to recover azadirachtin from neem seeds can include providing neem seeds, crushing the neem seeds, extracting azadirachtin from the crushed seeds with water, and then extracting azadirachtin from the water by adding a second extraction solvent including a non-aqueous solvent that is not miscible with water and has a higher solubility of azadirachtin than water or a surfactant having a turbidity temperature between 20 °C and 80 °C. The concentrated azadirachtin can be recovered from the second extraction solution and shows high activity as an insecticide and parasiticide. Extraction methods employing polar solvents (e.g. water) lead to extracts that are rich in polar components, such as azadirachtin compounds.

In various aspects, the azadirachtin suitable for use herein includes azadirachtin A, which is by its scientific name of dimethyl[2aR-[2aα,3β,4β(1aR*,2S*,3aS*,6aS*,7S*,7aS*),4aβ,5α, 7aS*,8β(E),10,1βaα,10bβ]]-10-(acetyloxy)octahydro-3,5-dihydroxy-4-methyl-8-[(2-methyl-1-oxo-2-butenyl)oxy]-4-(3a,6a,7,7a)-tetrahydro-6a-hydroxy-7a-methyl-2,7-methanofuro[2,3-b]oxireno[e]oxepin-1a(2H)-yl)-1H,7H-naphtho-[1,8-bc:4,4a-c′]difuran-5,10a(8H)-dicarboxylate.

Azadirachtin A is the most abundant of a group of the azadirachtinoids. Azadirachtin A makes up about 80% of the azadirachtinoids in the neem seed kernel. The structural formula of azadirachtin A is:

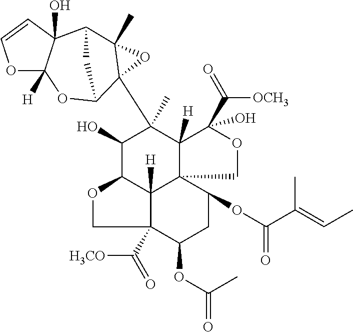

The pest control agents herein can include neem extracts that are an aqueous extract. In various aspects, the neem extract can include an aqueous extract of neem seed. The aqueous extract of neem seed can include an aqueous extract of the neem seed kernel. The aqueous extract of neem seed can include an aqueous extract of the entire neem seed, including the neem seed kernel and the neem seed coating. The aqueous extract of neem seed can be in liquid form, or it can be dried to remove water to create a powder form. By way of example, the neem extracts herein can include an aqueous extract of neem seed or an aqueous extract of neem seed kernel that has been dried into a powder.

It will be appreciated that the pest control agents described herein are not the same thing as neem extracts described as neem oil or solvent-first neem extracts. In various aspects, the pest control agents herein including azadirachtin are richer in the azadirachtinoid active ingredients, and in particular azadirachtin A, than are neem oil and other oil-based formulations. This is due to the fact that azadirachtinoids, such as azadirachtin A, are relatively polar complex terpenoids with a large number of oxygen functionalities, which make the molecules moderately water-soluble (e.g., a solubility of approximately 2 g/L). As a result, azadirachtinoids such as azadirachtin A are present in much higher concentrations in the extracts obtained employing polar solvents than in neem oil or solvent-first neem extracts. Without wishing to be bound by any particular theory, it is believed that the bioavailability of the active ingredients to the target parasite in the water-based extract of azadirachtin A rich extracts of the present disclosure can be greater than in neem oil given the increased solubility and miscibility of the water-based extract in water. Thus, the pest control agents herein do not, comprise, consist, or consist essentially of, neem oil. The pest control agent of the fish feed provided herein can comprise, consist, or consist essentially of azadirachtin A.

The pest control agents including neem extract rich in azadirachtin A can include those having from at least 15 wt. % to 33 wt. % azadirachtin A. In various aspects, pest control agents including neem extract rich in azadirachtin A can include those having from at least 20 wt. % to 26 wt. % azadirachtin A. In various aspects, pest control agents including neem extract rich in azadirachtin A can include those having from at least 28 wt. % to 31 wt. % azadirachtin A. In some aspects, pest control agents including neem extract rich in azadirachtin A can include those having from at least 29 wt. % to 30 wt. % azadirachtin A. In other aspects, pest control agents including neem extract rich in azadirachtin A can include those having from at least 34 wt. % to 40 wt. % azadirachtin A. In various aspects, pest control agents rich in azadirachtin A can include those having from 30±1 wt. % azadirachtin A. In various aspects, pest control agents rich in azadirachtin A can include those having from 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, or 33 wt. %, or any amount falling within a range of any of the forgoing. In yet other aspects, pest control agents including neem extract rich in azadirachtin A can include those having from at least 34 wt. % to 45 wt. % azadirachtin A, or at least 38 wt. % to 43 wt. %. As used herein, the terms “neem extract rich in azadirachtin A” and “azadirachtin A-rich composition” can be used interchangeably unless otherwise noted. A composition of an exemplary neem extract rich in azadirachtin A pest control agent suitable for use herein can include the formula as outlined in Table 1.

TABLE 1
Exemplary Azadirachtin-A Rich Pest Control Agent Formulation

| Azadirachtin Composition | % w/w |
|---|---|
| Azadirachtin A | 15-33 |
| Azadirachtin B | ≤19.0 |
| Azadirachtin D | ≤13.0 |
| Azadirachtin E | ≤5.0 |
| Azadirachtin F | ≤5.0 |
| Azadirachtin G | ≤5.0 |
| Azadirachtin H | ≤5.0 |
| Azadirachtin I | ≤5.0 |
| Azadirachtin K and other Azadirachtins | ≤5.0 |
| Total Azadirachtins | 52.0 |

The pest control agent including a neem extract rich in azadirachtin A can further include other azadirachtinoids at various concentrations. The azadirachtinoids can include azadirachtin compounds such as azadirachtin B at from ≤19.0% w/w, or from ≤6.0% w/w, or from 4.0 to 6.0% w/w, or from 5.6% w/w to 6.0% w/w; azadirachtin D at from ≤13.0% w/w, or from ≤5.0% w/w, or from 2.5 to 5.0, or from 4.0% w/w to 5.0% w/w; azadirachtin E at from ≤5.0% w/w, or from 1.0% w/w to 5.0% w/w, or from 1.5% w/w to 2.0% w/w; azadirachtin F at from K 5.0% w/w, or from 1.0% w/w to 5.0% w/w, or from 1.5% w/w to 2.0% w/w; azadirachtin G at from ≤5.0% w/w, or from 1.0% w/w to 5.0% w/w, or from 1.5% w/w to 2.0% w/w; azadirachtin H at from K 5.0% w/w, or from 1.0% w/w to 5.0% w/w, or from 2.5% w/w to 4.0% w/w; azadirachtin I at from ≤5.0% w/w, or from 1.0% w/w to 4.0% w/w, or from 1.5% w/w to 2.5% w/w; and azadirachtin K and/or other azadirachtin variants at from ≤5.0% w/w, or from 1.0% w/w to 5.0% w/w, or from 2.5% w/w to 4.0% w/w. The extracts further can include azadirachtinin at from ≤5.0% w/w, or from 1.0% w/w to 5.0% w/w, or from 2.5% w/w to 4.0% w/w.

An exemplary pest control agent suitable for use herein can include an aqueous extract of neem seed that has been dried into a powder. The powder can include the appearance of a fine white powder. The exemplary pest control agent can include azadirachtin A at a concentration of from 17 wt. % to 37 wt. %, azadirachtin B at a concentration of from 0 wt. % to 19 wt. %, and azadirachtin D at a concentration of rom 0 wt. % to 13 wt. %. The exemplary pest control agent further can include trace amounts of other limonoids including nimbin and salannin.

Exemplary pest control agents including azadirachtin A rich compositions include, but are not to be limited to, NeemAzal® (Coromandel, Intl. Ltd., Telangana, India) or NeemAzal® Technical (Coromandel, Intl. Ltd., Telangana, India), or any derivatives of combinations thereof.

### Fish Feeds Containing Azadirachtin A

The fish feeds herein can include those that are supplemented with the pest control agent azadirachtin A. The fish feeds can be administered to various fish as part of a fish feed diet to control non-copepod parasitic pests within an aquaculture environment. The fish feeds can be at least partially coated on an exterior surface with an azadirachtin A-rich composition or completely coated on an exterior surface with an azadirachtin A-rich composition. In some aspects, the fish feeds herein can include an azadirachtin A-rich composition that is at least partially dispersed throughout the fish feed. In various aspects, a solid feed such as a base feed pellet, can further include an azadirachtin A-rich composition disposed on the surface or distributed throughout the fish feed, such as within an oil disposed within a porous matrix of on an exterior surface of the base feed pellet. In various aspects, the fish feeds herein can be at least partially coated on an exterior surface with an azadirachtin A-rich composition and further can have an azadirachtin A-rich composition at least partially dispersed throughout the fish feed. In various aspects, the fish feed can include one or more layers of azadirachtin A-rich composition on an exterior surface.

The fish feeds herein can include an azadirachtin A-rich composition at a concentration from about 0.01-100 grams per kilogram (g/kg) fish feed, about 90 g/kg fish feed, about 80 g/kg fish feed, about 70 g/kg fish feed, about 60 g/kg fish feed, about 50 g/kg fish feed, about 40 g/kg fish feed, about 30 g/kg fish feed, about 20 g/kg fish feed, about 0.01-10 g/kg fish feed, about 1-10 g/kg fish feed, about 2-9 g/kg fish feed, about 3-7 g/kg fish feed, about 4-6 g/kg fish feed, or about 5 g/kg fish feed.

In various aspects, the fish feeds herein can include an azadirachtin A-rich composition at a concentration from about 0.01 g azadirachtin A-rich composition per kilogram fish feed (g/kg), 0.05 g/kg fish feed, 0.1 g/kg fish feed, 0.2 g/kg fish feed, 0.3 g/kg fish feed, 0.4 g/kg fish feed, 0.5 g/kg fish feed, 0.6 g/kg fish feed, 0.7 g/kg fish feed, 0.8 g/kg fish feed, 0.9 g/kg fish feed, 1.0 g/kg fish feed. 1.25 g/kg fish feed, 1.5 g/kg fish feed, 1.75 g/kg fish feed, 2.0 g/kg fish feed, 2.25 g/kg fish feed, 2.5 g/kg fish feed, 2.75, g/kg fish feed, 3.0 g/kg fish feed, 5.0 g/kg fish feed. 5.25 g/kg fish feed, 5.5 g/kg fish feed, 5.75 g/kg fish feed, 6.0 g/kg fish feed, 6.25 g/kg fish feed, 6.5 g/kg fish feed, 6.75, g/kg fish feed, 7.0 g/kg fish feed, 7.0 g/kg fish feed. 7.25 g/kg fish feed, 7.5 g/kg fish feed, 7.75 g/kg fish feed, 8.0 g/kg fish feed, 8.25 g/kg fish feed, 8.5 g/kg fish feed, 8.75, g/kg fish feed, 9.0 g/kg fish feed, 9.25 g/kg fish feed, 9.5 g/kg fish feed, 9.75, g/kg fish feed, 10.0 g/kg fish feed, 15 g/kg fish feed, 20 g/kg fish feed, 25 g/kg fish feed, 30 g/kg fish feed, 35 g/kg fish feed, 40 g/kg fish feed, 45 g/kg fish feed, 50 g/kg fish feed, 55 g/kg fish feed, 60 g/kg fish feed, 65 g/kg fish feed, 70 g/kg fish feed, 75 g/kg fish feed, 80 g/kg fish feed, 85 g/kg fish feed, 90 g/kg fish feed, 95 g/kg fish feed, 100 g/kg fish feed, or any amount within a range of any of the forgoing concentrations.

It will be appreciated that the aforementioned azadirachtin A-rich composition concentrations equate to about from 0.001-10 (weight percent) % w/w azadirachtin A-rich composition to fish feed. In various aspects, the fish feeds herein can include an azadirachtin A-rich composition at from 0.001% w/w, 0.002% w/w, 0.003% w/w, 0.004% w/w, 0.005% w/w, 0.006% w/w, 0.007% w/w, 0.008% w/w, 0.009% w/w, 0.010% w/w, 0.020% w/w, 0.030% w/w, 0.040% w/w, 0.050% w/w, 0.060% w/w, 0.070% w/w, 0.080% w/w, 0.090% w/w, 0.10% w/w, 0.11% w/w, 0.12% w/w, 0.13% w/w, 0.14% w/w, 0.15% w/w, 0.16% w/w, 0.17% w/w, 0.18% w/w, 0.19% w/w, 0.20% w/w, 0.21% w/w, 0.22% w/w, 0.23% w/w, 0.24% w/w, 0.25% w/w, 0.26% w/w, 0.27% w/w, 0.28% w/w, 0.29% w/w, 0.30% w/w, 0.31% w/w, 0.32% w/w, 0.33% w/w, 0.34% w/w, 0.35% w/w, 0.36% w/w, 0.37% w/w, 0.38% w/w, 0.39% w/w, 0.40% w/w, 0.41% w/w, 0.42% w/w, 0.43% w/w, 0.44% w/w, 0.45% w/w, 0.46% w/w, 0.47% w/w, 0.48% w/w, 0.49% w/w, 0.50% w/w, 0.51% w/w, 0.52% w/w, 0.53% w/w, 0.54% w/w, 0.55% w/w, 0.56% w/w, 0.57% w/w, 0.58% w/w, 0.59% w/w, 0.60% w/w, 0.61% w/w, 0.62% w/w, 0.63% w/w, 0.64% w/w, 0.65% w/w, 0.66% w/w, 0.67% w/w, 0.68% w/w, 0.69% w/w, 0.70% w/w, 0.71% w/w, 0.72% w/w, 0.73% w/w, 0.74% w/w, 0.75% w/w, 0.76% w/w, 0.77% w/w, 0.78% w/w, 0.79% w/w, 0.80% w/w, 0.81% w/w, 0.82% w/w, 0.83% w/w, 0.84% w/w, 0.85% w/w, 0.86% w/w, 0.87% w/w, 0.88% w/w, 0.89% w/w, 0.90% w/w, 0.91% w/w, 0.92% w/w, 0.93% w/w, 0.94% w/w, 0.95% w/w, 0.96% w/w, 0.97% w/w, 0.98% w/w, 0.99% w/w, 1.0% w/w, 2.0% w/w, 3.0% w/w, 4.0% w/w, 5.0% w/w, 6.0% w/w, 7.0% w/w, 8.0% w/w, 9.0% w/w, or 10.0% w/w (i.e., weight azadirachtin A-rich composition to weight fish feed), or any amount within a range of any of the forgoing values.

In various aspects herein, the fish feeds can be administered to the fish having an azadirachtin A-rich composition at a concentration selected from the group including 0.05% w/w, 0.06% w/w, 0.07% w/w, 0.08% w/w, 0.09% w/w, 0.10% w/w, 0.11% w/w, 0.12% w/w, 0.13% w/w, 0.14% w/w, 0.15% w/w, 0.16% w/w, 0.17% w/w, 0.18% w/w, 0.19% w/w, 0.20% w/w, 0.30% w/w, 0.40% w/w, 0.50% w/w, 0.60% w/w, 0.70% w/w, 0.80% w/w, 0.90% w/w, or 1.0% w/w (i.e., weight azadirachtin A-rich composition to weight fish feed), or a range within any of the forgoing concentrations.

Azadirachtins can be relatively unstable in water, however when they are a component of a fish feed, such as dispersed throughout or coated thereon, the azadirachtins, including azadirachtin A, are rendered at least temporarily stable such that they can exhibit their full biological activity during feeding. Any fish feed that falls to the ocean floor will degrade upon prolonged exposure to water. In addition, it should be noted that azadirachtin A, or any of the neem extract agents of the present disclosure exhibit minimal risk of toxic effects on fish or humans and are therefore safe to use in both wild fish and farmed fish stocks. While arthropods and other invertebrates are sensitive to the active ingredient (i.e. azadirachtin A), higher organisms, including mammals, are unaffected. Furthermore, since azadirachtin A or any neem extract as described herein are readily soluble in water, they do not reside and accumulate in fish. Rather, once administration has ceased, the pest control agent can quickly lose effectiveness, as it is metabolized, degraded, and/or excreted. In the case of farmed fish stocks, this ensures a little to no withdrawal period to harvest following administration of a fish feed or composition as described herein.

The fish feed provided herein can include an azadirachtin A-rich composition together with one or more other agents. The one or more or other agents can include anti-ectoparasitic agents, antimicrobial agents (e.g., antibacterial, antifungal, antiviral agents), antiparasitic agents (e.g., anti-endoparasitic agents or anti-ectoparasitic agents), or antiprotozoal agents. The one or more other agents can be mixed with or coated on, or layered within, the fish feed. The one or more other agents can be provided separately (e.g., either in liquid or solid form) and can be administered separately (e.g., before or after) or concurrently with (e.g., during) a fish feed.

### Immunomodulatory Effects of Administering Dietary Pest Control Agents

The pest control agents herein can exert one or more immunomodulatory effects in fish infested with the pests described herein. In various aspects, the pest control agents herein can exert an immunomodulatory effect by lessening or removing any dysregulation of the immune system as experienced by the fish. The dysregulation of the immune system by the parasites as described can include the suppression of protective, beneficial immune responses in parallel with the exaggeration of non-protective or harmful immune responses. In various aspects, the use of the pest control agents herein can exert an immunomodulatory effect by reducing, controlling, or preventing the parasitic load on the animal, where the parasitic load can include the number of parasites present during the infection or infestation. By reducing the parasitic load, the pest control agents can reduce undesirable inflammatory responses left otherwise unchecked by the immune system in response to the infection or infestation, or can stimulate protective immune protective and physiological responses. Left unchecked, an overactivated immune system can cause a number of types of damage to the host fish's tissues. As such, in some aspects, the reduction of the undesirable inflammatory responses can be an indirect effect due to the reduction of the number of parasites on the fish. Similarly, stimulation of protective immune responses and physiological responses can also be a result of direct or indirect effects of the pest control agents on parasites or the host.

In various aspects, the reduction of undesirable inflammatory responses can be a direct response to the pest control agent itself. In other aspects, the pest control agents can exert an immunomodulatory effect by reducing, controlling, or preventing the infection or infestation with a pest such that there is no suppression of protective, beneficial immune responses and no upregulation of non-protective or harmful immune responses, thus allowing the immune system to function optimally. It will be appreciated that beneficial protective immune responses can include recognition of parasitic organisms by the immune system, activation of protective immune responses and protective physiological responses, repair of damage caused by non-protective immune responses, and the subsequent removal of parasites. The harmful non-protective immune responses can include those that can lead to immunopathology, such as tissue damage causes by dysregulated immune responses. The harmful non-protective immune responses can also include those that negatively affect the health of the fish by stopping, preventing, or decreasing the effectiveness of the protective immune responses when the fish are faced with an immune challenge, such as an overactivation of regulatory and anti-inflammatory pathways or an activation of competing, non-protective immune responses induced by parasites. In various aspects, the immunomodulatory effect exerted by the pest control agent can include reducing, controlling, or preventing an infection or infestation, thereby minimizing negative effects of the infection or infestation, including minimizing excess mortality, minimizing deleterious effects on flesh quality, and minimizing a decrease in animal welfare, and allowing fish to fight off other pathogens and stressors including heat stress, hypoxic stress, oxidative stress, etc., as compared to infected or infested fish not administered the pest control agent.

### Method of Making a Fish Feed

The disclosure herein provides a method of making a fish feed including one or more pest control agents, such as neem extracts including azadirachtin A-rich compositions. The method can include the step of providing a base feed and applying a quantity of pest control agent to a surface of the base feed. A base feed can be formed from various raw materials as described elsewhere herein. For example, the method can include coating the base feed with a quantity of pest control agent. The base feeds can be at least partially coated on an exterior surface with a pest control agent or completely coated on an exterior surface with a pest control agent. In some aspects, the base feeds herein can include a pest control agent that is at least partially dispersed throughout the base feed. In various aspects, the base feeds herein can be at least partially coated with a pest control agent and further can have a pest control agent at least partially dispersed throughout the base feed. In some aspects, the pest control agent can be distributed throughout the base feed, such as throughout a porous matrix of the base feed. In various aspects, the base feed can be coated on an exterior surface with more than one layer of pest control agent, where each layer can include the same pest control agent, or in some aspects each layer can include a different pest control agent.

The pest control agent can be incorporated into or mixed into the base feed by various processes. In various aspects, the base feed can be made using an extrusion process or a pressing process. The pest control agent can be mixed with the base during its manufacture such that it becomes distributed through all or a part of the fish feed. Once the pest control agent has been mixed with the base feed, the base feed and pest control agent mixture can be formed into, for example, pellets, flakes, tablets, powders, or any form as desired. In the case of temperature sensitive pest control agents, such pest control agents can be added to a base feed after it has been formed into one of the various forms as indicated. In various aspects, the pest control agent can be sprayed onto a base feed that has already been formed into pellets, flakes, tablets, and the like. For example, the pest control agent can be applied to a base feed as one or more layers or top coatings. In various aspects, the pest control agent can be applied to an outside surface of a pellet or a flake—in this way a fish feed pellet or flake can become wholly or partially coated with the pest control agent. One or more layers or coatings of agent can be applied to an outside surface of a fish feed flake or pellets. Any layer or coating of agent can be “sealed” or protected by the application of one or more additional coatings or layers of a sealing substance. In various aspects, the pest control agent herein can be dispersed in one or more oils or fractions thereof and can be incorporated into a porous matrix within the fish feed by a vacuum coating process.

By way of example, a layer or coating of agent can be sealed by the application of a layer or coating of oil, such as fish oil. In various aspects, one or more further layers or coatings of fish feed can be applied to the (optionally sealed) coating or layer of fish feed. In this way, any given fish feed flake or pellet can include multiple layers of fish feed, sealing substance and/or pest control agent layers. It will be appreciated that the fish feeds described herein can be at least partially coated on an exterior surface with pest control agent. In various aspects, the fish feeds herein can include a pest control agent that is at least partially dispersed throughout the fish feed. In various aspects, the fish feeds herein can include a pest control agent that is at least partially coated with pest control agent and at least partially dispersed throughout the fish feed.

The method for incorporating the pest control agent into the fish feed can include incorporating the pest control agent, such that the final concentration of pest control agent in the fish feed includes from about 0.01 gram pest control agent per kilogram of fish feed (g/kg) to about 1000 g/kg, or from about 0.01 g/kg, 0.1 g/kg, 1 g/kg, 2 g/kg, 3 g/kg, 4 g/kg, 5 g/kg, 6 g/kg, 7 g/kg, 8 g/kg, 9 g/kg or 10 g/kg, 20 g/kg, 30 g/kg, 40 g/kg, 50 g/kg, 60 g/kg, 70 g/kg, 80 g/kg, 90 g/kg, 100 g/kg, 250 g/kg, 500 g/kg, 750 g/kg, or 1000 g/kg, or any amount within a range of any of the forgoing. Concentrations are described herein in more detail in reference to the fish feed.

The method for incorporating the pest control agent into the fish feed can include determining the final concentration of pest control agent that is incorporated as a part of the fish feed. The determination of the final concentration can include sampling the fish feed using various quantitative analytical methods. By way of example, the fish feed samples can be extracted by a process of overnight protein precipitation in methanol. Following extraction the sample can be cleaned with Supel™ QuE Z-Sep+ (Sigma Aldrich, St. Louis, Missouri, USA) sorbent, which is a silica gel-based material having active zirconia-based phase, a particle size of approximately 50 μm, and a 70-angstrom (A) pore size. The resulting extraction solution can be filtered through polytetrafluoroethylene filters having a pore size from 0.2 μm or greater. Analysis of the final concentration of pest control agent in the fish feed samples can be performed using high performance liquid chromatography with ultraviolet detection (IPLC-UV).

The methods herein can include extracting a neem extract rich in azadirachtin A, including those having at least from 15 wt. % to 33 wt. % azadirachtin A, from at least 28 wt. % to 31 wt. % azadirachtin A, and from at least 29 wt. % to 30 wt. % azadirachtin A.

The method further can include the step of sealing the azadirachtin A-rich composition applied to a surface of the fish feed. The azadirachtin A-rich composition can be sealed by applying a coating of fish oil to the azadirachtin A-rich composition coated fish feed. Any sealing substance used to seal the azadirachtin A-rich composition can be applied such that it coats all or a part of the azadirachtin A-rich composition coating.

### Pest Control Agent Compositions

The present disclosure further provides a pest control agent composition for administration to fish, where the pest control agent composition can include one or more pest control agents. It will be appreciated that the pest control agent compositions are not a fish feed and are intended for separate or supplemental administration to fish in addition to a fish feed. The pest control agent compositions can be provided separately for administration before, during, or after administration of the fish feeds. Accordingly, in various aspects, the pest control agent compositions herein can be suitable for use in some aspects as a form of veterinary medicinal product or dietary supplement for reducing, preventing, or controlling pest infections or infestations in fish. The pest control agent compositions can be administered to the fish at the concentrations described elsewhere herein. For example, the pest control agent compositions herein can be administered at from about 0.01 grams pest control agent per kilogram of fish feed (g/kg) to about 100 g/kg, as described elsewhere herein. In various aspects, the pest control agent composition includes a neem extract rich in azadirachtin A.

The pest control agent compositions herein can include azadirachtin extracts rich in azadirachtin A. The pest control agent composition can include a liquid, solid, or semi-solid form, and further can include one or more of an excipient, diluent, carrier, vitamins, minerals, or combinations thereof. The pest control agent compositions can be in the form of a dietary supplement that is provided as any of granules, flakes, pellets, powders, tablets, pills, capsules, and the like. In various aspects, the pest control agent compositions herein can be formed into many shapes and sizes. In various aspects, the fish feeds herein can be in the shape of a triangle, a square, a rectangle, a sphere, a diamond, a cylinder, a pellet, a clover, an amorphous shape, and the like. The fish feeds can be formed by a process including one or more of extrusion, retort, cold-pressing, high-pressure processing, and the like.

Alternatively, the pest control agent composition can be provided in a form that is edible by fish but that does not provide nutrition to the fish. By way of example, the pest control agent composition can include a veterinary medicinal product that can include substances or combinations of substances to manage or prevent diseases in fish. The pest control agent composition can also be formulated for parenteral administration. Thus, the pest control agent composition can include pharmaceutically acceptable carriers, diluents, or excipients, or combinations thereof. Furthermore, the pest control agent composition can be sterile.

The pest control agent compositions herein can be included in one or more types of fish feed designed for mixing with another composition, such as a base feed. The pest control agent composition can be in the form of a premix, a concentrate, a base mix, a supplement, a top dress, liquid drench, or a combination thereof.

The pest control agent in the pest control agent compositions herein can include one or more agents for reducing, preventing, or controlling an infection or infestation caused or contributed by one or more pests, including any type of worms, helminths, flukes, bacteria, viruses, fungi, and protozoa, as described elsewhere. Each pest control agent included in the pest control agent compositions can be individually capable of reducing, preventing, or controlling one or more of a parasitic, bacterial, viral, fungal, or protozoal infections or infestations. By way of example, the pest control agent compositions herein can include those exhibiting one or more inhibitory effects, including an antiparasitic effect, an antibacterial effect, an antiviral effect, an antifungal effect, an antiprotozoal effect, or any combinations thereof.

A pest control agent composition can be administered before during or after the administration of any of the fish feeds. In some aspects, the pest control agent compositions can be administered with fish feed that does not contain a pest control agent. In some aspects, the pest control agent compositions can be administered in conjunction with fish feed that does contain a pest control agent. When used in conjunction with fish feed that does contain a pest control agent, the separate pest control agent composition can include the same pest control agent as in the fish feed or it can be a different pest control agent than in the fish feed. When used in conjunction with fish feed that does contain a pest control agent, the separate pest control agent composition can be the same concentration as the pest control agent in the fish feed or it can be a different concentration than the pest control agent in the fish feed. The pest control agent compositions herein can be included in the diet of fish in the form of a veterinary medicinal product or dietary supplement to any complete and balanced fish feed or can be provided as a component of a complete fish feed.

### Methods of Administration of Pest Control Agents to Fish

The pest control agents herein can be administered to fish in the fish feeds and pest control agent compositions. Management methods that utilize the pest control agents within fish feeds can be referred to as in-feed agent delivery methods. Thus, the present disclosure provides in-feed agent delivery methods for reducing, preventing, or controlling pests. It will be appreciated that an in-feed agent delivery method is not a process that applies the pest control agents topically to the target pests. Management methods herein can further utilize pest control agents in a non-feed form such as a veterinary medicinal product or dietary supplement. Thus, the present disclosure further provides veterinary medicinal products or dietary supplements as agents for reducing, preventing, or controlling pests.

The pest control agents that are not included in fish feed can be administered to the fish in a separate pest control agent composition as a complement to fish feed, such as in the form of a veterinary medicinal product or a dietary supplement. The fish feed can be administered at the same time or separately from a pest control agent composition. It should be noted that the various pest control agents herein can be administered to fish that are sick, fish that are infested with parasites, fish that are otherwise healthy in order to prevent parasitic infection, or fish that are less aggressive due to a different infection or condition not associated with a pest infection or infestation. It will be understood that fish that are sick or less aggressive may eat less and therefore may consume lower concentrations of the pest control agent. Thus, management methods that are based on the use of both fish feeds and pest control agent compositions can be particularly useful for managing fish whose appetites are affected by illness, infection, infestation, or being a less aggressive fish that generally eats less fish feed. Moreover, in less aggressive, or low feeding fish, the concurrent use of a pest control agent composition with fish feed supplemented with a pest control agent can boost or ensure the correct concentration of pest control agent is administered to fish.

The concentration of pest control agent added to fish feeds or pest control agent compositions herein can be an amount effective to achieve the desired modulation of the behavior, development, or mortality of the pests as discussed elsewhere herein. It will be appreciated that the exact amount of pest control agent to be added to a fish feed or pest control agent compositions herein can vary depending on, for example, the species of fish, the number of fish to be fed, the extent of the infection or infestation, and the like. Other factors that influence the amount of pest control agent added to the fish feeds or pest control agent compositions include, for example, the presence of possible competitors for the feed (i.e. other non-target animal species that can eat the fish feed), the type of pest to be controlled, the age and maturity of the pests, the age and maturity of the fish, the season, the water type (e.g., pH, salinity, purity, temperature), and the aggressiveness of the fish. It will be appreciated that the concentration of pest control agents added to a fish feed or pest control agent composition herein can include an amount effective to achieve a desired effect to modulate the behavior and development of the pests, where the amount effective includes one or more concentrations or ranges of concentrations as recited herein. It will be appreciated that the effective amount effective can be determined by performing a comparison to a control fish or group of fish not fed the pest control agents.

The fish feeds and pest control agent compositions can be formulated such that the concentration of the pest control agent administered to the fish through the fish feed or pest control agent compositions can be approximately 0.01-100 mg pest control agent per kg body weight/day (mg/kg/day), 1-90 mg/kg/day, 1-80 mg/kg/day, 1-70 mg/kg/day, 1-60 mg/kg/day, 5-50 mg/kg/day, 10-40 mg/kg/day, 15-35 mg/kg/day, 20-30 mg/kg/day, 0.01-10 mg/kg/day, or 0.01-5.0 mg/kg/day. In various aspects, the pest control agent is administered to the fish in the fish feed at a targeted concentration from 0.01 mg to 5.0 mg azadirachtin A per kg body weight per day. In various aspects, the pest control agent is administered to the fish in the fish feed at a targeted concentration from 1.5 mg to 2.5 mg azadirachtin A per kg body weight per day. In some aspects, the pest control agent is administered to the fish in the fish feed at a targeted concentration from 2.6 mg to 5.0 mg azadirachtin A per kg body weight per day.

It will be appreciated that the amount of pest control agent administered to the fish can include an amount effective to produce an inhibitory effect against one or more pests within a range of approximately 0.01-100 mg/kg/day (e.g., mg pest control agent/kg body weight/day) includes at least 0.01 mg/kg/day, 0.02 mg/kg/day, 0.03 mg/kg/day, 0.04 mg/kg/day, 0.05 mg/kg/day, 0.06 mg/kg/day, 0.07 mg/kg/day, 0.08 mg/kg/day, 0.09 mg/kg/day, 0.10 mg/kg/day, 0.20 mg/kg/day, 0.30 mg/kg/day, 0.40 mg/kg/day, 0.50 mg/kg/day, 0.60 mg/kg/day, 0.70 mg/kg/day, 0.80 mg/kg/day, 0.90 mg/kg/day, 1.0 mg/kg/day, 2.0 mg/kg/day, 3.0 mg/kg/day, 4.0 mg/kg/day, 5.0 mg/kg/day, 6.0 mg/kg/day, 7.0 mg/kg/day, 8.0 mg/kg/day, 9.0 mg/kg/day, 10.0 mg/kg/day, 11.0 mg/kg/day, 12.0 mg/kg/day, 13.0 mg/kg/day, 14.0 mg/kg/day, 15.0 mg/kg/day, 16.0 mg/kg/day, 17.0 mg/kg/day, 18.0 mg/kg/day, 19.0 mg/kg/day, 20.0 mg/kg/day, 25.0 mg/kg/day, 30.0 mg/kg/day, 35.0 mg/kg/day, 40.0 mg/kg/day, 45.0 mg/kg/day, 50.0 mg/kg/day, 55.0 mg/kg/day, 60.0 mg/kg/day, 65.0 mg/kg/day, 70.0 mg/kg/day, 75.0 mg/kg/day, 80.0 mg/kg/day, 85.0 mg/kg/day, 90.0 mg/kg/day, 95.0 mg/kg/day, or 100.0 mg/kg/day, or any amount within a range of any of the forgoing values. Any of the aforementioned amounts effective to produce an inhibitory effect can be utilized in the targeted concentrations described herein.

A fish feed or pest control agent composition can be administered for a period of time for as long as required to achieve the desired inhibitory effect. For example, the pest control agent composition or fish feed can be administered over about a 10 to 20 days, or about 14-18 days. It will be appreciated that, the pest control agent composition, the fish feed, or both, can be administered for consecutive days for 10 days, 11 days, 12 days, 13 days, 14 days, 15 days, 16 days, 17 days, 18 days, 19 days, or 20 days, or for any number of days falling within a range of any of the forgoing. In various aspects, the pest control agent composition or fish feeds herein can be administered for a longer period of time, such as past 20 days. In various aspects, the fish feed or pest control agent can be administered for at least 11 days. In various aspects, the fish feed or pest control agent can be administered for at least 14 days. It should be understood that the time required for administration of the pest control agent composition or fish feeds herein can be of a variable length in order to target the developmental life stages of the pests present in a fish population, for water temperature, pest control agent concentration, or any combinations thereof. In some aspects, the pest control agents herein could be administered prophylactically in the diet of fish at an amount effective to prevent a pest infection or infestation from taking hold within a population of fish. It will further be appreciated that the pest control agents herein could be administered prophylactically in the diet for any period of time during the fish life cycle, such as from stocking to harvest, seasonally, or during an infection or infestation outbreak within a population or within a nearby farm infection or infestation outbreak.

In various management methods, the pest control agents herein can be administered for non-consecutive days, where the pest control agent is administered for a predetermined period of time followed by a rest period, and then administered again for a predetermined period of time and followed by a rest period, and so on. By way of example, in some aspects, the pest control agent can be administered for three out of every 10 days. In other aspects, the pest control agent can be administered for seven out of every 14 days. The method for administering the pest control agent for predetermined period of time followed by a rest period can be repeated for as long as desired or until a pest infection or infestation is reduced, prevented, or controlled. It will be appreciated that the pest control agent can be administered for a predetermined period of time, including from 1 day, 2 days, 3 days, 4 days, 5 days, 6 days, 7 days, 8 days, 9 days, or 10 days out of every 5 days to 30 days of rest in between administration.

During the period of administration, the pest control agent composition or fish feed can be administered as many times per day as required to achieve the inhibitory effect. For example, the pest control agent composition or fish feeds described herein can be administered about 1, 2, 3, 4, 5, 6, 7, 8, 9, 10, or more times a day.

Administration of the pest control agents herein to fish using the fish feeds and pest control agent compositions can be performed by various methods. In an aspect, a method for reducing, preventing, or controlling a non-copepod parasitic infection or infestation in a fish population. The method can include providing a fish feed including a neem extract rich in azadirachtin A, the neem extract including from 15 wt. % to 33 wt. % of azadirachtin A. The method can include administering to one or more fish the fish feed including the neem extract rich in azadirachtin A, where the fish feed provides a concentration from 0.01 mg to 5.0 mg azadirachtin A per kg body weight per day to the one or more fish.

In an aspect, a method for reducing, preventing, or controlling a non-copepod parasitic infection or infestation in fish is provided. The method can include providing a pest control agent composition including a pest control agent including neem extract rich in azadirachtin A. The method can include administering the pest control agent composition to one or more fish for from 1 to 20 days during an infection or infestation, where the concentration of azadirachtin A administered to the fish through the pest control agent composition is from about 0.01 mg to 5 mg azadirachtin A per kg body weight per day.

For the methods herein, any method can be directed at administering the fish feed to fish or fish populations experiencing a non-copepod parasitic infection or infestation that is caused by one or more organisms including Amyloodinium ocellatum, Cardicola forsteri, Cryptocaryon irritans, Diplectanum spp., Gyrodactylus salaris, Henneguya salminicola, Ichthyophthirius multifiliis, Kudoa thyrsites, Myxobolus cerebralis, Neoparamoeba perurans, Saprolegnia parasitica, Sparicotyle chrysophrii, Trichodina spp., or Zeylanicobdella arugamensis.

For the methods herein, any method can include where the concentration of azadirachtin A includes a concentration in an amount effective to increase efficacy of azadirachtin A against a non-copepod parasitic infection or infestation as compared to fish fed a diet lacking the neem extract rich in azadirachtin A.

For the methods herein, any method can include administering the fish feed to the farmed fish for at least 14 days.

For the methods herein, any method can include where the neem extract rich in azadirachtin A is administered to the fish at a concentration from 1.5 mg to 2.5 mg azadirachtin A per kg body weight per day.

For the methods herein, any method can include where the neem extract rich in azadirachtin A is administered to the fish at a concentration from 2.6 mg to 5.0 mg azadirachtin A per kg body weight per day.

For the methods herein, any method can include where the fish feed further includes one or more components including antibacterial agents, antifungal agents, antiviral agents, antiparasitic agents, or antiprotozoal agents.

For the methods herein, any method can include where the fish feed is administered to species of fish belonging to one or more families including Cyprinidae, Cichlidae, Pangasiidae, Sciaenidae, Serranidae, Carangidae, Sparidae, Lateolabracidae, Moronidae, Mugilidae, Cypriniformes, Latidae, Eleotridae, Tilapiini, and Salmonidae.

For the methods herein, any method can include where the pest control agent is configured to produce an inhibitory effect including one or more of an antiparasitic effect, an antibacterial effect, an antiviral effect, an antifungal effect, or an antiprotozoal effect.

For the methods herein, any method can include where the neem extract rich in azadirachtin A does not include neem oil.

For the methods herein, any method can include where the pest control agent is provided in the fish feed to the pests in an amount sufficient to modulate the behavior of the pests.

For the methods herein, any method can include where the modulating the behavior of the pests includes one or more of a change in feeding habits, a change in feeding patterns, a change in appetite, a change in mobility patterns, or a change in mating patterns as compared to pests found on control animals not fed a pest control agent.

For the methods herein, any method can include where the neem extract rich in azadirachtin A is obtained by a method including the steps of providing neem seeds; crushing the neem seeds; extracting azadirachtin from the crushed seeds with water; adding a second extraction solution that includes a non-aqueous solvent which is not miscible with water and has a higher solubility of azadirachtin than water or a surfactant having a turbidity temperature between 20 °C and 80 °C; and recovering the concentrated azadirachtin from the second extraction solution.

### Targeted Management Methods for Pest Control Agent Administration

The methods for administering the pest control agents herein can include targeted management methods that utilize a targeted concentration and a targeted duration of exposure to maximize the antiparasitic effects of the pest control agent against various life stages of the pests. The targeted management methods can include administering a pest control agent through one or more fish feeds or pest control agent compositions as described elsewhere herein. The targeted concentration can include an amount effective to produce an inhibitory effect against one or more pests, where the inhibitory effect can include one or more of an anti-feedancy effect, an anti-molting effect, an antifertility and anti-fecundity effect, or an antiparasitic effect. The targeted duration of exposure can include a period of time that is required to achieve the desired inhibitory effect. It will be appreciated that in various aspects, some life stages will require a smaller targeted concentration and targeted duration of exposure, while other life stages will require a larger targeted concentration and longer targeted duration of exposure.

By targeting the concentration and the duration of exposure at various life stages, the management methods can be tailored to maximize efficacy at the lowest concentration of pest control agent required to elicit an inhibitory effect. By utilizing the lowest concentration of pest control agent possible, the management methods can achieve high efficacy at lower targeted concentrations in order to keep the costs of management low for farmers. As described elsewhere herein, the pest control agents can include those neem extracts rich in azadirachtin A that can produce inhibitory effects against one or more pests including one or more effects for reducing, preventing, or controlling the concentration and spread of various parasites. As described, azadirachtin A has anti-feedant properties against pests, where the anti-feedant properties can exert one or more anti-feedancy effects including malnourishment, delayed development, prevention or delay of molting, and death, all of which can contributed to excess mortality of the pests at all life stages over time.

The inhibitory effect can include a reduction in the total number of pests available to molt from one developmental life stage to another as compared to a population fish infected or infested with one or more pests that are fed a diet lacking the neem extract rich in azadirachtin A. The inhibitory effect can further include a reduction in the total number of adult females or a reduction in total number of adult males in a pest population exposed to fish fed a diet containing azadirachtin A as compared to a population fish infected or infested with one or more pests that are fed a diet lacking the neem extract rich in azadirachtin A.

The inhibitory effect of the pest control agents can lead to excess mortality in the pest population. For example, pest mortality can increase with each day that the pests are exposed to the targeted concentrations during the targeted duration of exposure and is referred to herein as “extra daily mortality.” Pest mortality also increases at each molting stage and is referred to herein as “extra mortality at molting.” In addition to causing extra daily mortality due to malnourishment, the neem extracts rich in azadirachtin A can substantially inhibit or prevent the development of pests from one stage to the next to result in extra mortality at molting. Further, the neem extracts rich in azadirachtin A can exhibit an ovicidal effect on the female pests, where the ovicidal effect can include a decrease in egg production, a decrease in egg viability, a decrease in egg size, and a decrease in time to hatching to further lead to extra mortality at molting.

Targeted management methods of the present disclosure can be tailored to expose the pests to pest control agent within one or more development windows to target various or multiple life stages. It will be appreciated that the developmental life stage composition of pest populations present during an infection or infestation of farmed fish changes in response to various environmental factors, including the season, ocean currents, water temperature, or the use of physical delousing systems. The targeted management methods herein can include alternating between the administration of the pest control agent at the lower range of targeted concentrations and at a higher range of targeted concentrations, depending on the life stage present at a given time. Further, the methods herein can include administering the pest control agents at the onset infection or infestation, part way through the infection or infestation, or later in the infection or infestation, or combinations thereof. It will be appreciated that the onset of the infection or infestation can include from days 0 to 9 post infection with the pests; part way through the infection or infestation can include from days 10 to 19 post infection with the pests; and later in the infection or infestation can include from days 20 days or more post infection with the pests.

By way of example, some methods can include alternating between administering targeted concentrations from 0.01 mg/kg fish/day to 2.5 mg/kg fish/day for 14 days to target the early life stages of the pests, followed by administering targeted concentrations from 2.6 mg/kg fish/day to 5.0 mg/kg fish/day for 14 days to also target the later life stages of the pests.

In some aspects of the methods herein, a first management method including an exposure to pest control agent can be immediately followed by a second management method including an exposure to a pest control agent, while in other aspects a first management method including an exposure can be followed by a second management method including an exposure to a pest control agent after waiting a predetermined period of time. The predetermined period of time can include from 1 day to 60 days, or any number of days falling within a range from 1 day to 60 days. In various aspects, the predetermined period of time can include more than 60 days. It will be appreciated that during the predetermined period of time, the fish exposed to pest control agent can be allowed to rest and recover from any external stresses that were placed upon them during either of the first or second management methods. In some aspects, the methods herein can include repeating a cycle of a first exposure to a pest control agent and a second exposure to a pest control agent for as long as necessary to reduce or prevent an infection or infestation with the pests described herein. In various aspects, a third exposure to a pest control agent, different than the first or second exposures to a pest control agent, can be used, where the third exposure to a pest control agent includes a concentration of pest control agent that is different than either of the first management method or second management method.

It should be understood that the definitions described herein apply to all aspects as described unless otherwise stated.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference is to be considered supplementary to that of this document; for irreconcilable inconsistencies, the usage in this document controls.

Values expressed in a range format are to be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” is to be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis (% w/w or w/w %) is also referred to as weight percent (wt. %) or percent by weight (% wt.) herein.

Exemplary embodiments of the present invention are as follows:

Embodiment 1: A method for reducing, preventing, or controlling a non-copepod parasitic infection or infestation in a fish population comprising:
providing a fish feed comprising a neem extract rich in azadirachtin A, the neem extract comprising from 15 wt. % to 33 wt. % of azadirachtin A;
administering to one or more fish the fish feed comprising the neem extract rich in azadirachtin A; and
wherein the fish feed provides a concentration from 0.01 mg to 5.0 mg azadirachtin A per kg body weight per day to the one or more fish.

Embodiment 2: The method of Embodiment 1, wherein the non-copepod parasitic infection or infestation is caused by one or more parasitic organisms comprising Amyloodinium ocellatum, Cardicola forsteri, Cryptocaryon irritans, Diplectanum spp., Gyrodactylus salaris, Henneguya salminicola, Ichthyophthirius multifilis, Kudoa thyrsites, Myxobolus cerebralis, Neoparamoeba perurans, Saprolegnia parasitica, Sparicotyle chrysophrii, Trichodina spp., or Zeylanicobdella arugamensis.

Embodiment 3: The method of Embodiment 1, wherein the fish feed is administered to the farmed fish for at least 11 days.

Embodiment 4: The method of Embodiment 1, wherein the fish feed is administered to the farmed fish for at least 14 days.

Embodiment 5: The method of Embodiment 1, wherein the concentration of azadirachtin A comprises a concentration in an amount effective to increase efficacy of azadirachtin A against a non-copepod parasitic infection or infestation as compared to fish fed a diet lacking the neem extract rich in azadirachtin A.

Embodiment 6: The method of any of Embodiments 1-5, wherein the neem extract rich in azadirachtin A is administered to the fish at a concentration from 1.5 mg to 2.5 mg azadirachtin A per kg body weight per day.

Embodiment 7: The method of any of Embodiments 1-6, wherein the neem extract rich in azadirachtin A is administered to the fish at a concentration from 2.6 mg to 5.0 mg azadirachtin A per kg body weight per day.

Embodiment 8: The method of any of Embodiments 1-7, wherein the fish feed further comprises one or more components comprising antibacterial agents, antifungal agents, antiviral agents, antiparasitic agents, or antiprotozoal agents.

Embodiment 9: The method of any of Embodiments 1-8, wherein the fish feed is administered to species of fish belonging to one or more families comprising Cyprinidae, Cichlidae, Pangasiidae, Sciaenidae, Serranidae, Carangidae, Sparidae, Lateolabracidae, Moronidae, Mugilidae, Cypriniformes, Latidae, Eleotridae, Tilapiini, and Salmonidae.

Embodiment 10: The method of any of Embodiments 1-9, wherein the pest control agent is configured to produce an inhibitory effect comprising one or more of an antiparasitic effect, an antibacterial effect, an antiviral effect, an antifungal effect, or an antiprotozoal effect.

Embodiment 11: The method of any of Embodiments 1-10, wherein the neem extract rich in azadirachtin A does not comprise neem oil.

Embodiment 12: The method of any of Embodiments 1-11, wherein the pest control agent is provided in the fish feed to the pests in an amount sufficient to modulate the behavior of the pests.

Embodiment 13: The method of any of Embodiments 1-12, wherein modulating the behavior of the pests comprises one or more of a change in feeding habits, a change in feeding patterns, a change in appetite, a change in mobility patterns, or a change in mating patterns as compared to pests found on control animals not fed a pest control agent.

Embodiment 14: The method of Embodiment 12, wherein the change in feeding patterns comprises a decrease in feeding patterns.

Embodiment 15: The method of Embodiment 12, wherein the change in appetite comprises a decrease in appetite.

Embodiment 16: The method of Embodiment 12, wherein the change in mobility comprises a decrease in mobility.

Embodiment 17: The method of Embodiment 12, wherein the change in mating patterns comprises a decrease in mating patterns.

Embodiment 18: The method of Embodiment 17, wherein the change in mating patterns comprises a decrease in the number of viable pests present on the fish or in the fish habitat as compared to pests found on control fish not fed a pest control agent.

Embodiment 19: The method of any of Embodiments 1-18, wherein the neem extract rich in azadirachtin A is obtained by a method comprising the steps of
providing neem seeds;
crushing the neem seeds;
extracting azadirachtin from the crushed seeds with water;
adding a second extraction solution that comprises:
   a non-aqueous solvent which is not miscible with water and has a higher solubility of azadirachtin than water; or
   a surfactant having a turbidity temperature between 20 °C and 80 °C; and
recovering the concentrated azadirachtin from the second extraction solution.

Embodiment 20: A method for reducing, preventing, or controlling a non-copepod parasitic infection or infestation in fish comprising:
providing a pest control agent composition comprising a pest control agent comprising neem extract rich in azadirachtin A; and
administering the pest control agent composition to one or more fish for from 1 to 20 days during an infection or infestation;
wherein the concentration of azadirachtin A administered to the fish through the pest control agent composition is from 0.01 mg to 5 mg azadirachtin A per kg body weight per day.

1.-15. (canceled)

16. A method for reducing, preventing, or controlling a non-copepod parasitic infection or infestation in a fish population comprising:
providing a fish feed comprising a neem extract rich in azadirachtin A, the neem extract comprising from 15 wt. % to 33 wt. % of azadirachtin A;
administering to one or more fish the fish feed comprising the neem extract rich in azadirachtin A; and
wherein the fish feed provides a concentration from 0.01 mg to 5.0 mg azadirachtin A per kg body weight per day to the one or more fish.

17. The method of claim 16, wherein the non-copepod parasitic infection or infestation is caused by one or more parasitic organisms comprising Amyloodinium ocellatum, Cardicola forsteri, Cryptocaryon irritans, Diplectanum spp., Gyrodactylus salaris, Henneguya salminicola, Ichthyophthirius multifiliis, Kudoa thyrsites, Myxobolus cerebralis, Neoparamoeba perurans, Saprolegnia parasitica, Sparicotyle chrysophrii, Trichodina spp., or Zeylanicobdella arugamensis.

18. The method of claim 16, wherein the fish feed is administered to the farmed fish for at least 11 days.

19. The method of claim 16, wherein the fish feed is administered to the farmed fish for at least 14 days.

20. The method of claim 16, wherein the concentration of azadirachtin A comprises a concentration in an amount effective to increase efficacy of azadirachtin A against a non-copepod parasitic infection or infestation as compared to fish fed a diet lacking the neem extract rich in azadirachtin A.

21. The method of claim 16, wherein the neem extract rich in azadirachtin A is administered to the fish at a concentration from 1.5 mg to 2.5 mg azadirachtin A per kg body weight per day.

22. The method of claim 16, wherein the neem extract rich in azadirachtin A is administered to the fish at a concentration from 2.6 mg to 5.0 mg azadirachtin A per kg body weight per day.

23. The method of claim 16, wherein the fish feed further comprises one or more components comprising antibacterial agents, antifungal agents, antiviral agents, antiparasitic agents, or antiprotozoal agents.

24. The method of claim 16, wherein the fish feed is administered to species of fish belonging to one or more families comprising Cyprinidae, Cichlidae, Pangasiidae, Sciaenidae, Serranidae, Carangidae, Sparidae, Lateolabracidae, Moronidae, Mugilidae, Cypriniformes, Latidae, Eleotridae, Tilapiini, and Salmonidae.

25. The method of claim 16, wherein the pest control agent is configured to produce an inhibitory effect comprising one or more of an antiparasitic effect, an antibacterial effect, an antiviral effect, an antifungal effect, or an antiprotozoal effect.

26. The method of claim 16, wherein the neem extract rich in azadirachtin A does not comprise neem oil.

27. The method of claim 16, wherein the pest control agent is provided in the fish feed to the pests in an amount sufficient to modulate the behavior of the pests.

28. The method of claim 16, wherein modulating the behavior of the pests comprises one or more of a change in feeding habits, a change in feeding patterns, a change in appetite, a change in mobility patterns, or a change in mating patterns as compared to pests found on control animals not fed a pest control agent.

29. The method of claim 28, wherein the change in feeding patterns comprises a decrease in feeding patterns.

30. The method of claim 28, wherein the change in appetite comprises a decrease in appetite.

31. The method of claim 28, wherein the change in mobility comprises a decrease in mobility.

32. The method of claim 28, wherein the change in mating patterns comprises a decrease in mating patterns.

33. The method of claim 32, wherein the change in mating patterns comprises a decrease in the number of viable pests present on the fish or in the fish habitat as compared to pests found on control fish not fed a pest control agent.

34. The method of claim 16, wherein the neem extract rich in azadirachtin A is obtained by a method comprising the steps of:
providing neem seeds;
crushing the neem seeds;
extracting azadirachtin from the crushed seeds with water;
adding a second extraction solution that comprises:
a non-aqueous solvent which is not miscible with water and has a higher solubility of azadirachtin than water; or
a surfactant having a turbidity temperature between 20 °C and 80 °C; and
recovering the concentrated azadirachtin from the second extraction solution.

35. A method for reducing, preventing, or controlling a non-copepod parasitic infection or infestation in fish comprising:
providing a pest control agent composition comprising a pest control agent comprising neem extract rich in azadirachtin A; and
administering the pest control agent composition to one or more fish for from 1 to 20 days during an infection or infestation;
wherein the concentration of azadirachtin A administered to the fish through the pest control agent composition is from 0.01 mg to 5 mg azadirachtin A per kg body weight per day.
